(19) United States
(12) Patent Application Publication
(10) Pub. No.: US 2014/0161785 A1
(43) Pub. Date: Jun. 12, 2014

(54) Verticillin A Inhibition of Histone Methyltransferases

(71) Applicants: Feiyan Liu, Hangzhou (CN); Ping Wu, Hangzhou (CN); Kebin Liu, Martinez, GA (US)

(72) Inventors: Feiyan Liu, Hangzhou (CN); Ping Wu, Hangzhou (CN); Kebin Liu, Martinez, GA (US)

(21) Appl. No.: 14/098,797

(22) Filed: Dec. 6, 2013

Related U.S. Application Data
(60) Provisional application No. 61/735,258, filed on Dec. 10, 2012.

(51) Int. Cl. A61K 31/548; C12N 9/99; A61K 45/06

(52) U.S. Cl. CPC A61K 31/548; A61K 45/06; C12N 9/99
USPC 424/94.4; 514/222.8; 435/184

(57) ABSTRACT

Verticillin A is found to be a potent inhibitor of histone methyltransferases, selective for G9a, GLP, SUV39H1, SUV39H2, MLL1, and NSD2. Methods of using Verticillin A are provided. The Verticillin A can be synthetically produced or it can be isolated from natural sources. Methods if inhibiting one or more histone methyltransferases are provided. In addition, methods are provided for treating diseases or disorders related to overexpression of one or more histone methyltransferases. Exemplary diseases and disorders to be treated include cancer, asthma, HIV, and progeria.

### CROSS-REFERENCE TO RELATED APPLICATIONS

This application claims priority to and the benefit of U.S. Provisional Application No. 61/735,258, filed Dec. 10, 2012.

### STATEMENT REGARDING FEDERALLY SPONSORED RESEARCH OR DEVELOPMENT

This invention was made with government support under Agreement R01 CA133085 by the National Institutes of Health, and Agreement RSG-09-209-TBG by the American Cancer Society. The government has certain rights in the invention.

### FIELD OF THE INVENTION

This invention is generally related to methods of inhibiting histone methyltransferases in a subject using for example, Verticillin A or a derivative thereof.

### BACKGROUND OF THE INVENTION

The preceding decade of biomedical research has resulted in new understandings and insight into many of the chemical modifications of histone proteins that are critical for gene regulation. As an example of relevance here, it is now well appreciated that the histone proteins exhibit specific posttranslational modifications with rich information content. These histone modifications form a sort of “histone code” that plays a central role in regulating gene expression. As insight grows into these epigenetic mechanisms, it is having a profound impact on translational biology. Recent data suggests that epigenetic changes in normal tissue may precede and predispose one to cancer, similar to the way that altered lipid metabolism may predispose one to heart disease decades before it becomes symptomatic. For other diseases there is a growing consensus that epigenetic changes (allele-specific silencing, methylation, chromatin modification) play a major role. Heritable epigenetic traits appear to play a role in neuropsychiatric disease, bipolar disease, and autism.

The development of drugs that modify histone markers has largely targeted the histone acetlytransferases and histone deacetylases. These enzymes add or remove acetyl groups on specific conserved lysine amino acids in certain histone proteins. This research has resulted in new pharmaceuticals such as the histone deacetylase inhibitor Vorinostat, marketed under the trade name ZOLINA® for treating certain cancers. Acetylation and methylation are two of the most prominent posttranslational modifications of the histones that control gene expression. Therefore, active agents that can target histone methyltransferases and histone demethylases provide an important next avenue in drug development.

It is therefore an object of the invention to identify compounds that are inhibitors of histone methyltransferases.

It is a further object of the invention to identify compounds that are potent inhibitors specific for one or a few histone methyltransferases.

It is a further object of the invention to provide methods of selectively inhibiting one or a few histone methyltransferases.

It is a further object of the invention to provide methods of using histone methyltransferase inhibitors to treat diseases or disorders that are related to the methylation of DNA-binding proteins.

It is yet another object of the invention to provide methods and compositions for modulating the methylation of DNA-binding proteins.

### SUMMARY OF THE INVENTION

It has been discovered that Verticillin A is a potent and selective histone methyltransferase inhibitor. Verticillin A, the structure of which is shown below, has been found to selectively inhibit the following methyltransferases: G9a, GLP, SUV39H1, SUV39H2, MLL1, and NSD2.

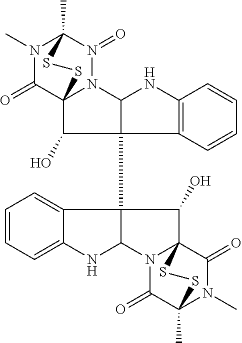

Verticillin A was observed to have potent inhibitory activity for the enzymes G9a (IC₅₀ of 0.54 μM), SUV39H1 (IC₅₀ of 0.57 μM), and SUV39H2 (IC₅₀ of 0.48 μM). Verticillin A was observed to also exhibit inhibitory activity against MLL1 (IC₅₀ of 3.08 μM) NSD2 (IC₅₀ of 4.13 μM), and GLP (IC₅₀ of 1.27 μM).

Methods are provided for the inhibition of one or more histone methyltransferases by contacting the methyltransferase with an effective amount of Verticillin A. Diseases and disorders are described that may be treated by the inhibition of one or more of these histone methyltransferases, and methods are provided for treating a patient in need thereof. Exemplary diseases or disorders that may be treated with Verticillin A include leukemia, latent HIV, asthma, and progeria.

In some embodiments a method is provided for modulating the methylation of DNA-binding proteins, for example histone proteins, by administering to a subject in need thereof, an effective amount of Verticillin A. Preferably, methylation of the DNA-binding proteins is reduced after administration of Verticillin A.

Some embodiments provide methods for treating cancer by administering to a subject in need thereof an effective amount of Verticillin A. In some embodiments the administration of Verticillin A results in slowing the growth and progression of tumor cells.

Some embodiments provide methods for inducing latent viral reservoirs by administering to a subject in need thereof an effective amount of Verticillin A. Preferably, administration of Verticillin A results in induction of latent virus. In some embodiments the virus is human immunodeficiency virus (HIV) and the method includes providing to a subject in need thereof a therapeutically effective amount of Verticillin A, optionally in combination with one or more antiretroviral drugs.

Some embodiments provide methods for treating asthma by administering to a subject in need thereof a therapeutically effective amount of Verticillin A. Preferably, the administration of Verticillin A provides for down-regulation of Th2 cells.

In some embodiments methods are provided for slowing the progression of cellular senescence by administering to a subject in need thereof a therapeutically effective amount of Verticillin A. In certain embodiments the subject in need thereof has progeria. Preferably, the administration of Verticillin A results in extending the lifespan of subjects with progeria.

### BRIEF DESCRIPTION OF THE DRAWINGS

FIG. 1 is a graph depicting the percentage of enzyme activity relative to the activity in DMSO as a function of the concentration of Verticillin A (M) for the methyltransferases EZH1, G9a, GLP, SUV39H1, SUV39H2, MLL1, and NSD2. The determined IC₅₀ values (μM) are included in parentheses in the legend.

FIG. 2 depicts images of Western blotting analysis performed in human colon carcinoma cells (LS411N in the left panels and SW620 in the right panels). The histone-lysine marks measured include the di-methylation of histone-3-lysine-9 (H3K9Me2, panels A1 and A2), the tri-methylation of histone-3-lysine-9 (H3K9Me3, panels B1 and B2), and the tri-methylation of histone-3-lysine-27 (H3K27Me3, panels C1 and C2). Histone-3 (H3, panels D1 and D2) is used as a normalization control.

### DETAILED DESCRIPTION OF THE INVENTION

It has been discovered that Verticillin A is a potent and selective histone methyltransferase inhibitor. As discussed in more detail below, histones play an important role in regulating gene expression. The expression of certain histone modifications has been implicated in a broad range of diseases and disorders. Specific diseases and disorders are discussed. Formulations containing a therapeutically effective amount of Verticillin A and methods of treating specific diseases and disorders are described in detail below.

### I. Histones and Transcription

Histones are the highly alkaline chief protein components of chromatin found in eukaryotic cell nuclei. They are among the most highly conserved protein structure of eukaryotes, forming the “spools,” nucleosomes, around which the DNA in the nucleus winds. In recent years it has become appreciated that histones play an important role in regulating DNA transcription.

The histones have been classified into five major families. The families H2A, H2B, H3, and H4 constitute the core histones, while the family of H1/H5 histones is known as the linker histones. Each nucleosome core comprises 8 histones, two of each of the core histones, around which the DNA winds. The linker histones thereby sit on top keeping the DNA in place and linking the nucleosomes to form higher-order structures.

The term “Histone H2A” is a non-specific term that refers to a variety of closely related proteins that typically vary often by only a few amino acids. These can be further classified by the subfamilies Histone H2A F, H2A 1, and H2A 2 and include the specific histones H2AFB1, H2AFB2, H2AFB3, H2AFJ, H2AFV, H2AFX, H2AFY, H2AFY2, H2AFZ, HIST1H2AA, HIST1H2AB, HIST1H2AC, HIST1H2AD, HIST1H2AE, HIST1H2AG, HIST1H2AI, HIST1H2AJ, HIST1H2AK, HIST1H2AL, HIST1H2AM, HIST2H2AA3, and HIST2H2AC. H2A consists of a main globular domain and a long N-terminal tail or C-terminal on one end of the molecule. The N-terminal tail or C-terminal tail is the common location of post-translational modification.

The term “Histone H2B” also refers to a variety of closely related proteins further classified by the subfamilies H2BF, H2B1, and H2B2 and includes the specific exemplary histone proteins H2BFM, H2BFS, H2BFWT, HIST1H2BA, HIST1H2BB, HIST1H2BC, HIST1H2BD, HIST1H2BE, HIST1H2BF, HIST1H2BG, HIST1H2BH, HIST1H2BI, HIST1H2BJ, HIST1H2BK, HIST1H2BL, HIST1H2BM, HIST1H2BN, HIST1H2BO, and HIST2H2BE.

The term “Histone H3” refers to one of the five main families of histone proteins and represents the most extensively modified of the histone proteins by post-translational modifications. Specific subfamilies of histone H3 include H3A1, H3A2, and H3A3 and include the exemplary H3 histones HIST1H3A, HIST1H3B, HIST1H3C, HIST1H3D, HIST1H3E, HIST1H3F, HIST1H3G, HIST1H3H, HIST1H3I, HIST1H3J, HIST2H3C, and HIST3H3. As the most modified of the histone proteins, H3 histones have emerged to play an important role in gene regulation and the emerging science of epigenetics.

Featuring a main globular domain and a long N terminal tail, the “Histone H4” family comprises the families H41 and H44 histones and includes the exemplary histones HIST1H4A, HIST1H4B, HIST1H4C, HIST1H4D, HIST1H4E, HIST1H4F, HIST1H4G, HIST1H4H, HIST1H4I, HIST1H4J, HIST1H4K, HIST1H4L, and HIST4H4.

Each of the histone proteins may be post-transnationally modified in a number of ways; including through acetylation and methylation. These modifications alter their interaction with DNA and nuclear proteins. The long N-terminal and C-terminal tails can be covalently modified in a number of ways at several places. Modifications of the tail include methylation, acetylation, phosphorylation, ubiquitination, SUMOylation, citrullination, and ADP-ribosylation. Additionally, the core of the histones H2A, H2B, and H3 can also be modified. Histone modifications are typically identified using a code consisting of the name of the histone, the single-letter abbreviation of the amino acid modified followed by the position, and followed then by the type of modification. Phosphorylation is indicated by a P, acetylation by Ac, and methylation by Me followed by the number of methyl groups involved (1-3). As an example H3K9Me2 denotes di-methylization of lysine 9 in histone H3. This nomenclature will be employed throughout when needed.

Histone modifications impact the activity of a variety of biological processes such as gene regulation, DNA repair, chromosome condensation (mitosis) and spermatogenesis (meiosis). Histone lysine methylation can either activate or repress transcription dependent upon the specific combination of posttranslational marks presented. Elucidating the histone code remains a major challenge. With regards to histone methylation, it appears that methylation marks H3K4, H3K36, and H3K79 generally leads to activation whereas methylated H3K9, H3K27, and H4K20 marks appear to be repressive (C. Martin and Y. Zhang, Nature Reviews: Molecular and Cell Biology, 2005, 6, 838-849). The loss of the H4K20Me3 is generally associated with human cancers, typically associated with the additional loss of the H4K16Ac mark (M. F. Fraga et al., Nature Genetics, 2005, 37, 391-400).

### II. Histone Methyltransferases (HMTases)

Histone methyltransferases (HMTases) are histone-modifying enzymes that catalyze the transfer of methyl groups (one, two, or three may be transferred) from a methyl source to the lysine and arginine residues of histone proteins. More than 30 histone methyltransferases have been identified already(C. Qian and M. M. Zhou, Cellular Molecular Life Science, 2006, 63, 2755-2763; M. T. Bedford, Journal of Cell Science., 2007,120, 4243-4246). While some HMTases may exhibit methylation activity for both lysine and arginine, most are classified as either histone-lysine N-methyltransferase or histone-arginine N-methyltransferase. The most common proteins modified are the H3 and H4 histones. The histone methyltransferase cofactor S-adenosyl-methionine (SAM) is usually the methyl group donor.

The lysine-specific HMTases typically include one of two conserved structures, being classified as either SET (Su(var)3-9, Enhancer of Zeste, Trithorax) and non-SET domain containing HMTases. The non-SET domain containing HMTases typically utilize the Dot1 enzyme.

The lysine methyltransferase EZH2 has been observed to be overexpressed in a number of cancer types, including breast and prostate. SUV39H1 elevation has been associated with human colon cancer. Only a few small-molecule drug-like inhibitors of histone lysine methyltransferase that are both potent and selective have been developed. Owing to the unpredictable nature of the inhibitors, most have been obtained by random library screenings. The first such inhibitor was chaetocin, a fungal mycotoxin that was found to inhibit G9a, SUV39H1, and SUV39H2. The compounds Bix01294 and UNC0224 are selective for G9a, with UNC0224 showing nearly 1000-fold selectivity for G9a and an IC₅₀ of 0.015 μM. The structures of these inhibitors are shown below.

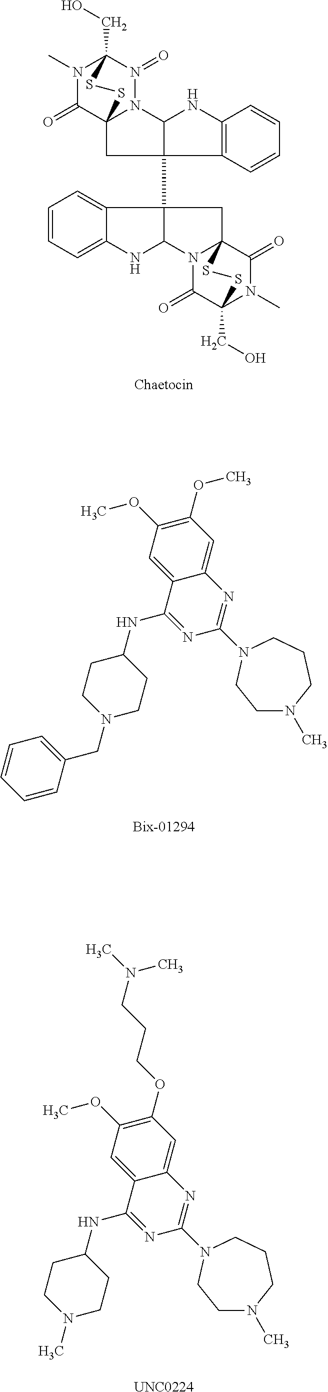

Unlike other post-translational modifications, methylation does not change the charge state of the nucleosome core. Methylation therefore is not expected to affect large-scale changes in the chromatin structure. Instead, the various methylation sites act as binding sites for other proteins through the highly-evolved methyl-lysine binding domains capable of differentiating between the specific lysine site, the degree of methylation, and often times the surrounding amino acid sequence. The histone lysine methylation can be associated with either transcriptional activation or repression. In one embodiment methods are provided for altering the expression of one or more genes using a histone methyltransferase inhibitor.

### III. Verticillin A

Verticillin A is a potent cytotoxin typically isolated from pathogen-infected poisonous mushrooms. The structure of Verticillin A is shown below.

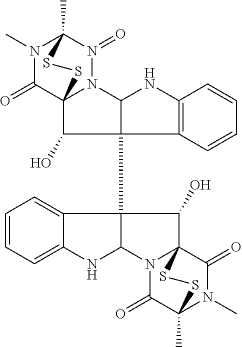

Verticillin A has been shown to be effective at overcoming resistance to apoptotics in certain cancers, including human colon cancer. Applicants have identified Verticillin A as a potent and selective inhibitor of certain histone methyltransferases discussed below. Based upon these findings, compositions, formulations, and methods of treatment are provided.

A. Verticillin A Inhibits Methyltransferases

Verticillin A was tested in Example 1 as an inhibitor against 18 known methyltransferases. In some embodiments contacting a methyltransferase with Verticillin A results in a greater than 50% reduction in methyltransferase activity, a greater than a 75% reduction in methyltransferase activity, preferably a greater than 90% reduction in methyltransferase activity, more preferably a greater than 99% reduction in methyltransferase activity, most preferably a greater than 99.9% reduction in methyltransferase activity. In some embodiments Verticillin A is a potent and selective inhibitor of only a few methyltransferases.

In some instances, different concentrations of Verticillin A may be required to achieve a given level of inhibitory activity for a given methyltransferase. In some embodiments, Verticillin A exhibits an IC₅₀ of less than 10 μM, preferably less than 5 μM, more preferably less than 1 μM, most preferably less than 0.6 μM for inhibition of one or in some cases more than one methyltransferase. In some embodiments Verticillin A inhibits the activity of one or more methyltransferases selected from the group consisting of G9a, GLP, SUV39H1, SUV39H2, MLL1, and NSD2. In some embodiments Verticillin A inhibits the activity of one or more methyltransferases selected from the group consisting of G9a, SUV39H1, and SUV39H2.

Verticillin A was tested in Example 2 for in vivo activity as a HMTase inhibitor in human colon carcinoma cells In some embodiments a dose of Verticillin A exhibits in vivo inhibitory activity for one or more histone-lysine methyl marks at a concentration of less than 200 nM, preferably less than 100 nm, preferably less than 90 nM, preferably less than 80 nM, preferably less than 70 nM, more preferably less than 60 nM, most preferably less than 50 nM. In some embodiments Verticillin A exhibits inhibition of one or more histone-lysine methyl marks at a concentration of 50 nM. In some instances Verticillin A exhibits in vivo inhibitory activity for one or more of the histone-lysine methyl marks selected from the group consisting of H3K9Me2, H3K9Me3, and H3K27Me3. In preferred embodiments Verticillin A exhibits in vivo inhibitory activity for H3K9Me2, H3K9Me3, and H3K27Me3.

In some embodiments methods are provided for modifying gene expression using Verticillin A by inhibiting or reducing methylation of DNA-binding proteins. In certain embodiments methods are provided for inhibiting or reducing the expression of one or more of the histone-lysine marks selected from the group consisting of H3K9Me2, H3K9Me3, and H3K27Me3. In some instances one or more histone methyltransferases are contacted with an effective amount of Verticillin A to inhibit the one or more histone methyltransferases. Preferred histone methyltransferases are selected from the group consisting of G9a, GLP, SUV39H1, SUV39H2, MLL1, and NSD2.

### IV. Compositions and Formulations

The administration of Verticillin A as a therapeutic agent for the treatment of a disease or disorder has broad applications because methyltransferases have been implicated in a variety of diseases and disorders. For example, Verticillin A can be used to treat cancer, asthma, and progeria. Verticillin A can also be used to induce latent viral reservoirs, in particular to induce latent reservoirs of HIV. Therefore, compositions containing Verticillin A are provided for treating various diseases and disorders.

Compositions may contain one or more additional active agents. Additional active agents may include cancer therapeutics, asthma therapeutics, or HIV therapeutics. In other embodiments, the additional active agents are administered in separate compositions, either simultaneously or at different times.

A. Verticillin A

Compositions containing a therapeutically effective amount of Verticillin A are provided for several treatments. In some embodiments, compositions are provided containing a therapeutically effective amount of an analog, derivative, salt, or prodrug of Verticillin A. The therapeutically effective amount may vary dependent upon several conditions including but certainly not limited to the disease or disorder being treated, the level of progression of the disease in the patient to be treated, the efficacy of additional active agents when provided in combination, and the overall age and weight of the patient.

Verticillin A may be synthetic or may be purified from natural sources. Verticillin A may be a single enantiomer or a mixture of enantiomers. In some embodiments the Verticillin A provided in the compositions is isolated from one or more natural sources. In some instances the Verticillin A is isolated from mushrooms. In other embodiments the Verticillin A is synthetic. Under some circumstances the purity of Verticillin A will be important. In some cases where the purity is important the Verticillin A is provided in the compositions with greater than 90% purity, preferably greater than 99% purity, more preferably greater than 99.9% purity, most preferably greater than 99.99% purity. Methods of purifying organic compounds are well known in the art.

B. Cancer Therapeutics

Compositions containing Verticillin A will in some embodiments contain one or more cancer therapeutics. In other instances compositions containing Verticillin A are administered in combination with one or more compositions containing one or more cancer therapeutics. The cancer therapeutics may include death receptor agonists such as the TNF-related apoptosis-inducing ligand (TRAIL) or Fas ligand or any ligand or antibody that binds or activates a death receptor or otherwise induces apoptosis. Suitable death receptors include, but are not limited to, TNFR1, Fas, DR3, DR4, DR5, DR6, LTβR and combinations thereof.

Conventional cancer therapeutics such as chemotherapeutic agents, cytokines, chemokines, and radiation therapy may be included. The majority of chemotherapeutic drugs can be divided in to: alkylating agents, antimetabolites, anthracyclines, plant alkaloids, topoisomerase inhibitors, and other antitumour agents. All of these drugs affect cell division or DNA synthesis and function in some way. Additional therapeutics include monoclonal antibodies and the new tyrosine kinase inhibitors e.g. imatinib mesylate (GLEEVEC® or GLIVEC®), which directly targets a molecular abnormality in certain types of cancer (chronic myelogenous leukemia, gastrointestinal stromal tumors).

Representative chemotherapeutic agents include, but are not limited to cisplatin, carboplatin, oxaliplatin, mechlorethamine, cyclophosphamide, chlorambucil, vincristine, vinblastine, vinorelbine, vindesine, taxol and derivatives thereof, irinotecan, topotecan, amsacrine, etoposide, etoposide phosphate, teniposide, epipodophyllotoxins, trastuzumab (HERCEPTIN®), cetuximab, and rituximab (RITUXAN® or MABTHERA®), bevacizumab (AVASTIN®), and combinations thereof.

C. HIV Therapeutics

In certain embodiments the composition containing Verticillin A also contains one or more conventional HIV therapeutics. In other embodiments a composition containing Verticillin A is administered in combination with one or more compositions containing one or more HIV therapeutics. An HIV therapeutic can be considered any therapeutic that restrains the growth and reproduction of the HIV. Suitable HIV therapeutics can include HIV protease inhibitors, HIV nucleoside and HIV nucleotide reverse-transcriptase inhibitors, and HIV non-nucleoside reverse transcriptase inhibitors. An HIV therapeutic may be an entry inhibitor that interferes with the initial binding, fusion, and entry of an HIV virion to the cell. Suitable entry inhibitors include the CCR5 receptor agonist Maraviroc (developed by Pfizer and marketed in the U.S. under the trade name SELZENTRY® by ViiV Healthcare) or Enfuvirtide (marketed under the trade name FUZEON® by Roche). Suitable HIV therapeutics may include one or more reverse transcriptase inhibitors (RTIs) that inhibit the copying of the viral genome by the reverse transcriptase in the cell. These may include nucleoside reverse transcriptase inhibitors (NRTIs), nucleotide reverse transcriptase inhibitors (NtRTIs), or non-nucleoside reverse transcriptase inhibitors (NNRTIs). Suitable NRTIs may include Zidovudine (marketed under the trade names of RETROVIR® and RETROVIS®), Didanosine, or Lamivudine (marketed under several trade names by GlaxoSmithKline). Suitable NtRTIs may include Tenofovir or the prodrug Tenofovir disoproxil fumarate (marketed under the trade name VIREAD® by Gilead Sciences) or Adefovir (marketed under the trade names PREVEON® and HEPSERA®). Suitable NNRTIs may include Efavirenz (marketed under multiple brand names), Nevirapine (marketed as VIRAMUNE® by Boehringer Ingelheim), or Rilpivirine (marketed as EDURANT® by Tibotec). Suitable HIV therapeutics may include protease inhibitors such as Saquinavir (marketed as FORTOVASE® by Hoffmann-La Roche), Ritonavir (marketed by Abbott Laboratories as NORVIR®), or Darunavir (marketed as PREZISTA® by Tibotec). HIV therapeutics may include integrase inhibitors, such as Elvitegravir, that interfere with the integration of the viral DNA into the cellular DNA. Suitable HIV therapeutics may also include what are known as maturation inhibitors, antiviral drugs that block or inhibit the virus maturation by binding the HIV gag protein. In some embodiments a composition containing Verticillin A contains a combination of multiple HIV therapeutics typically administered together in highly active antiretroviral therapy (HAART) treatment of HIV. Suitable combinations may include a combination of Zidovudine and Lamivudine or the combination of Abacavir and Lamivudine. In some instances a composition containing Verticillin A is administered in combination with a common fixed-dose HAART combination that may include the combination therapies marketed under the trade name COMBIVIR® by GlaxoSmithKline or one of the combination therapies marketed under the trade names TRUVADA® and STRIBILD® by Gilead Sciences.

D. Asthma Therapeutics

In certain embodiments a composition is provided containing Verticillin A in combination with one or more additional therapeutics that are conventionally used for treating patients with asthma. In other embodiments a composition containing Verticillin A is administered in combination with one or more conventional asthma therapeutics. Suitable asthma therapeutics may include corticosteroids such as fluticasone, budesonide, or ciclesonide. Suitable asthma therapeutics may include leukotriene modifiers or Leukotriene blockers such as montelukast (marketed as SINGULAIR®), zafirlukast (marketed as ACCOLATE®), or zileuton (marketed as ZYFLO®). The asthma therapeutic may in some cases be a long-acting beta agonist such as salmeterol (SEREVENT®) or formoterol (FORADIL® and PERFOROMIST®). In some cases a composition containing Verticillin A and one or more asthma therapeutics also contains one or more additional active agents selected from the group consisting of bronchodilators, vasodilators, and mucokinetics.

E. Additional Therapeutics

In some embodiments compositions containing Verticillin A are provided containing one or more additional therapeutics. The additional therapeutic may exhibit histone acetyltransferase (HAT) or histone deacetylase (HDAC) activity. In some instances the additional therapeutic may be a HAT inhibitor or an HDAC inhibitor. HDACs have been classified according to their homology to yeast HDACs. Class I, II, and IV HDACs are zinc-dependent enzymes while class III HDACs are NAD+ dependent enzymes. The HATs are typically grouped into two major classes; type A that include GCN-5 related N-acetyl transferases, p300/CBPm and MYST; and type B that are primarily associated with acetylation of histones prior to nucleosome formation. The epigenetic therapeutic may be a known HDAC, a known HAT, or a suitable derivative, salt, or prodrug thereof.

Several class I (HDAC1, HDAC2, HDAC3, HDAC8) and class II (HDAC4, HDAC5, HDAC6, HDAC7, HDAC9, HDAC10) HDAC inhibitors have passed or are currently in clinical trials. Exemplary HDAC inhibitors can include, but are not limited to, the hydroxamates such as Vorinostat (suberoylanilide hydroxamic acid, marketed under the trade name ZOLINZA® by Merck & Co. Inc. for T cell lymphoma) or the benzamides such as Entinostat (SNDX-275 and MS-275, currently in phase 2 clinical trials for treating non-small cell lung cancers and Hodgkin's lymphoma, Syndax Pharmaceuticals) or Mocetinostat (MGCD0103, completed phase 2 trials for Hodgkin's lymphoma, MethylGene Inc.) An HDAC inhibitor under clinical trials for treating T-cell lymphomas is Romidepsin (trade name ISTODAX® by Celgene Corporation). Suitable hydroxomate (hydroxamic acids) HDAC inhibitors could be understood to include those described in U.S. Pat. No. 6,455,531 B1 and 6,566,384 B1. An exemplary hydroxamic acid like inhibitor currently undergoing clinical trials for treating several malignant diseases (phase III), breast and prostate cancer (phase II), and leukemia (phase I) is Panobinostat. Class I and Class II HDAC inhibitors have been developed that are selective for HDAC1/HDAC2, that are selective for HDAC3, that are selective for HDAC4, that are selective for HDAC6, or that are selective for HDAC8. Examples of these are described for instance in the review article “Development of second generation epigenetic agents” written by Phillip Jones and published in. Medicinal Chemistry Communications, 2012, 3, 135-161 and the references cited therein. Class III HDACs include Sirtinol, Salermide, Splitomicin, and Cambinol.

The additional therapeutic may be a HAT inhibitor. The HAT inhibitor may be an isothiazolone compound that suppresses the activity of many HATs. The HAT inhibitor may be selective for suppressing the activity of one or more specific HATs. The HAT inhibitor may be a peptide conjugate of coenzyme A, such as the Lys-CoA conjugate that has shown potency and selectivity for p300/CBP or the H3-CoA-20 conjugate that has shown 100-fold selectivity for GCN-5. The HAT inhibitor may be a natural product such as curcumin, anacardic acid, or plumbagin, or a prodrug, salt, or derivative thereof. Many suitable HAT inhibitors are described for instance in “Development of second generation epigenetic agents” written by Phillip Jones and published in. Medicinal Chemistry Communications, 2012, 3, 135-161 and the references cited therein.

The additional therapeutic may exhibit methyltransferase (MTase) or demethylase (DMase) activity or may be a MTase of DMase inhibitor. In some embodiments the composition containing Verticillin A contains one or more additional HMTase inhibitors. The additional HMTase inhibitors may be inhibitors of the SET domain lysine HMTases, may be inhibitors of the non-SET domain lysine HMTases, or may be inhibitors of the arginine HMTases.

The one or more additional active agents include antibiotics. Exemplary antibiotics include aminoglycosides, cephalosporins, chloramphenicol, clindamycin, erythromycins, fluoroquinolones, macrolides, azolides, metronidazole, penicillins, tetracyclines, trimethoprim-sulfamethoxazole, or vancomycin.

The composition can contain one or more classes of narcotic and non-narcotic analgesics, such as morphine, codeine, heroin, hydromorphone, levorphanol, meperidine, methadone, oxydone, propoxyphene, fentanyl, naloxone, buprenorphine, butorphanol, nalbuphine, or pentazocine.

The composition can contain one or more classes of anti-inflammatory agents, including, but not limited to salicylates (such as acetylsalicylic acid, diflunisal and salsalate), propionic acid derivatives (such as ibuprofen, naproxen, fenoprofen, ketoprofen, flurbiprofen, oxaprozin, and ioxoprofen), acetic acid derivatives (such as indomethacin, sulindac, etodolac, and ketorolac), enolic acid (oxicam) derivatives (such as piroxicam, meloxicam, tenoxicam, droxicam, lornoxicam, and isoxicam), fenamic acid derivatives (such as mefenamic acid, meclofenamic acid, flufenamic acid, and tolfenamic acid), selective COX-2 inhibitors, sulphonanilides (such as nimesulide), and COX/LOX inhibitors (such as licofelone).

The composition can contain one or more classes of anti-histaminic agents, such as ethanolamines (e.g., diphenhydrmine carbinoxamine), ethylenediamines (e.g., tripelennamine pyrilamine), alkylamines (e.g., chlorpheniramine, dexchlorpheniramine, brompheniramine, triprolidine), or other anti-histamines such as astemizole, loratadine, fexofenadine, bropheniramine, clemastine, acetaminophen, pseudoephedrine, and triprolidine.

F. Pharmaceutical Formulations

The disclosed compositions can be formulated into suitable pharmaceutical preparations such as solutions, suspensions, tablets, dispersible tablets, pills, capsules, powders, delayed and/or sustained release formulations, or elixirs for oral administration, or in sterile solutions or suspensions for parenteral administration. In certain embodiments the disclosed compositions containing a therapeutically effective amount of Verticillin A are formulated into pharmaceutical compositions using techniques and procedures known in the art. It is understood that a therapeutically effective amount will depend upon multiple factors including but not limited to the type of formulation and route of administration; the specific disease or disorder to be treated; the presence, amount, and efficacy of additional therapeutic agents in the composition; among others.

In some embodiments, the disclosed compositions are formulated for single dosage administration. To formulate a composition, the weight fraction of active agent(s) is dissolved, suspended, dispersed or otherwise mixed in a selected carrier at an effective concentration such that the treated condition is relieved or one or more symptoms are ameliorated. The disclosed active agent(s) can be included in the pharmaceutically acceptable carrier in an amount sufficient to exert a therapeutically useful effect in the absence of undesirable side effects on the patient treated. The therapeutically effective concentration is determined empirically by testing the compounds in in vitro, ex vivo and in vivo systems, and then extrapolated therefrom for dosages for humans. The concentration of active agent(s) in the pharmaceutical composition will depend on absorption, inactivation and excretion rates of the active compound, the physicochemical characteristics of the agent, the dosage schedule, and amount administered as well as other factors known to those of skill in the art. Dosage forms or compositions containing active agent(s) in the range of 0.005% to 100% with the balance made up from non-toxic carrier may be prepared. Methods for preparation of these compositions are known to those skilled in the art. The contemplated compositions may contain 0.001%-100% active ingredient, or in one embodiment 0.1-95%.

Methods for solubilizing active agents or improving bioavailability may be used. Such methods are known to those of skill in this art, and include, but are not limited to, using cosolvents, such as dimethylsulfoxide (DMSO), using surfactants, such as TWEEN®, or dissolution in aqueous sodium bicarbonate. The pharmaceutical compositions of one or more of the active agents can be incorporated into a polymer matrix, for example, hydroxypropylmethyl cellulose, gel, permeable membrane, osmotic system, multilayer coating, microparticle, nanoparticle, liposome, microsphere, nanosphere, or the like. The active agent(s) may be suspended in micronized or other suitable form or may be derivatized (e.g., by adding one or more polyethylene glycol chains) to produce a more soluble active product or improve bioavailability. To optimize absorption, distribution, metabolism, and excretion, or improve oral bioavailability, the active agent(s) may be provided as prodrugs (i.e. in an inactive or significantly less active form which is metabolised in vivo into an active agent). The active agent(s) described herein may also be conjugated to a biomolecule, including, but not limited to a protein or nucleic acid, to affect bioavailability.

Liquid pharmaceutically administrable compositions can, for example, be prepared by dissolving, dispersing, or otherwise mixing the active agent(s) and optional pharmaceutical adjuvants in a carrier, such as, for example, water, saline, aqueous dextrose, glycerol, glycols, ethanol, and the like, to thereby form a solution or suspension. If desired, the pharmaceutical composition to be administered may also contain minor amounts of nontoxic auxiliary substances such as wetting agents, emulsifying agents, solubilizing agents, pH buffering agents and the like, for example, acetate, sodium citrate, cyclodextrin derivatives, sorbitan monolaurate, triethanolamine sodium acetate, triethanolamine oleate, and other such agents.

Oral pharmaceutical dosage forms can be either solid, gel, or liquid. The solid dosage forms can be tablets, capsules, granules, and bulk powders. Types of oral tablets include compressed, chewable lozenges and tablets which may be enteric-coated, sugar-coated or film-coated. Capsules may be hard or soft gelatin capsules, while granules and powders may be provided in non-effervescent or effervescent form with the combination of other ingredients known to those skilled in the art.

In certain embodiments, the formulations are solid dosage forms, in one embodiment, capsules or tablets. The tablets, pills, capsules, troches and the like can contain one or more of the following ingredients, or compounds of a similar nature: a binder; a lubricant; a diluent; a glidant; a disintegrating agent; a coloring agent; a sweetening agent; a flavoring agent; a wetting agent; an emetic coating; and a film coating.

The active agent(s), or a pharmaceutically acceptable salt(s) thereof, can be provided in a composition that protects it from the acidic environment of the stomach. For example, the composition can be formulated in an enteric coating that maintains its integrity in the stomach and releases the active compound in the intestine. The composition may also be formulated in combination with an antacid or other such ingredient.

When the dosage unit form is a capsule, it can contain, in addition to material of the above type, a liquid carrier such as a fatty oil. In addition, dosage unit forms can contain various other materials which modify the physical form of the dosage unit, for example, coatings of sugar and other enteric agents. In all embodiments, tablets and capsules formulations may be coated as known by those of skill in the art in order to modify or sustain dissolution of the active ingredient. Thus, for example, they may be coated with a conventional enterically digestible coating, such as phenylsalicylate, waxes, and cellulose acetate phthalate.

The pharmaceutical composition can be in a parenteral administration form. Injectables can be prepared in conventional forms, either as liquid solutions or suspensions, solid forms suitable for solution or suspension in liquid prior to injection, or as emulsions. The injectables, solutions, and emulsions may also contain one or more excipients. In addition, if desired, the pharmaceutical compositions to be administered may also contain minor amounts of non-toxic auxiliary substances such as wetting or emulsifying agents, pH buffering agents, stabilizers, solubility enhancers, and other such agents, such as for example, sodium acetate, sorbitan monolaurate, triethanolamine oleate and cyclodextrins. The percentage of active compound contained in such parenteral compositions is highly dependent on the specific nature thereof, as well as the activity of the compound and the needs of the subject.

Preparations for parenteral administration include sterile solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use and sterile emulsions. The solutions may be either aqueous or nonaqueous.

The unit-dose parenteral preparations can be packaged in an ampoule, a vial or a syringe with a needle. All preparations for parenteral administration should be sterile, as is known and practiced in the art. The injectable compositions described herein can be optimized for local and/or systemic administration.

The active agent(s) may be suspended in micronized or other suitable form. The active agent(s) may also be derivatized to produce a more soluble active product or to produce a prodrug. The form of the resulting mixture depends upon a number of factors, including the intended mode of administration and the solubility of the active agent(s) in the selected carrier or vehicle. The effective concentration is sufficient for ameliorating the symptoms of the condition and may be empirically determined.

Implantation of a slow-release or sustained-release system, such that a constant level of dosage is maintained is also contemplated herein. In such cases, the active agent(s) provided herein can be dispersed in a solid matrix optionally coated with an outer rate-controlling membrane. The compound diffuses from the solid matrix (and optionally through the outer membrane) sustained, rate-controlled release. The solid matrix and membrane may be formed from any suitable material known in the art including, but not limited to, polymers, bioerodible polymers, and hydrogels.

Lyophilized powders can be reconstituted for administration as solutions, emulsions and other mixtures. They may also be reconstituted and formulated as solids or gels. The sterile, lyophilized powder can be prepared by dissolving Verticillin A. The solvent may contain an excipient which improves the stability or other pharmacological component of the powder or reconstituted solution, prepared from the powder. The solvent may also contain a buffer, such as citrate, sodium or potassium phosphate or other such buffer known to those of skill in the art at. Subsequent sterile filtration of the solution followed by lyophilization under standard conditions known to those of skill in the art provides the desired formulation. In one embodiment, the resulting solution will be apportioned into vials for lyophilization. Each vial will contain a single dosage or multiple dosages of the compound. The lyophilized powder can be stored under appropriate conditions, such as at about 4° C. to room temperature. Reconstitution of this lyophilized powder with water for injection provides a formulation for use in parenteral administration. For reconstitution, the lyophilized powder is added to sterile water or other suitable carrier. The precise amount depends upon the selected compound. Such amount can be empirically determined.

The disclosed active agent(s), or pharmaceutically acceptable salts thereof, can be formulated to be targeted to a particular tissue, receptor, or other area of the body of the subject to be treated. Many such targeting methods are well known to those of skill in the art. In one embodiment, liposomal suspensions, including tissue-targeted liposomes, such as tumor-targeted liposomes, may also be suitable as pharmaceutically acceptable carriers. These may be prepared according to methods known to those skilled in the art. Compositions can be formulated to provide immediate or delayed release of one or more of the active agent(s), including Verticillin A. Also disclosed are sustained release formulations to maintain therapeutically effective amounts of Verticillin A over a period of time. In compositions containing multiple active agents, the active agents may be individually formulated to control the duration and/or time release of each active agent.

Such sustained and/or timed release formulations may be made by sustained release means of delivery devices that are well known to those of ordinary skill in the art. These pharmaceutical compositions can be used to provide slow or sustained release of one or more of the active agents using, for example, hydroxypropylmethyl cellulose, other polymer matrices, gels, permeable membranes, osmotic systems, multilayer coatings, microparticles, nanoparticles, liposomes, microspheres, nanospheres or the like. The active agents may also be suspended, micronized, or derivatized to vary release of the active ingredient(s).

### V. Methods of Use

The administration of Verticillin A as a therapeutic agent for the treatment of a disease or disorder has broad applications because methyltransferases have been implicated in a variety of diseases and disorders. For example, Verticillin A can be used to treat cancer, asthma, or progeria. In some embodiments, an analog, derivative, or prodrug of Verticillin A can be used to treat cancer, asthma, or progeria.

In some instances methods and compositions are provided for the treatments of diseases or disorders associated with overexpression of one or more HMTases. The compositions contain a therapeutically effective amount of Verticillin A, optionally containing one or more additional therapeutic agents. In other embodiments the compositions contain an analog, derivative, or prodrug of Verticillin A. In certain embodiments methods and compositions are provided for inhibiting the activity of one or more HMTases selected from the group consisting of MLL1, NSD2, GLP, G9a, SUV39H1, and SUV39H2. In preferred embodiments methods and compositions are provided for inhibiting the activity of one or more HMTases selected from the group consisting of G9a, SUV39H1, and SUV39H2. The method involves administering to a patient in need of inhibition or regulation of the activity of one or more HMTases a composition containing a therapeutically effective amount of Verticillin A.

A. Methods for Reprogramming Cells

Methods and compositions are provided for reprogramming cells without altering the underlying DNA sequence. The methods provided alter the rate of transcription of or the activation of specific genes. The method involves administering to a patient in need thereof a pharmaceutical formulation containing a therapeutically effective amount of Verticillin A. In certain embodiments the formulation containing a therapeutically effective amount of Verticillin A contains one or more additional therapeutics. The additional therapeutics will in some cases be selected from the group consisting of HATs, HDATs, HAT inhibitors, HDAT inhibitors, HMTases, HDMases, HMTase inhibitors, and HDMase inhibitors. In some cases the methods consists of administering to a patient in need thereof a formulation containing a therapeutically effective amount of Verticillin A in combination with one or more additional formulations containing one or more additional therapeutics. The formulations may be administered at the same time or at different times.

The therapeutically effective amount of Verticillin A for reprogramming cells will in some cases depend upon the specific gene or genes targeted and the levels present in the cell. In some embodiments a therapeutically effective amount of Verticillin A for reprogramming a cell will be an amount effective to result in a 2-fold reduction in gene activity, preferably a 10-fold reduction in gene activity, more preferably a 100-fold reduction in gene activity.

B. Methods for Treating Cancer

HMTases have been implicated in a number of human cancer cells. G9a and GLP have been observed to be up-regulated in various cancer cells (Huang et al. “G9a and GLP methylate lysine 373 in the tumor suppressor p53” J. Biol. Chem. 285:9636-9641, 2010). Knock-down of G9a suppresses tumor cell growth in vitro (Kondo et al., “Downregulation of histone H3 lysine 9 methyltransferase G9a induces centrosome disruption and chromosome instability in cancer cells”, 2008; Chen et al. “H3K9 histone methyltransferase G9a promotes lung cancer invasion and metastasis by silencing the cell adhesion molecule Ep-CAM” Cancer Res. 70:7830-7840, 2010). Therefore, in certain instances, compositions, formulations, and methods are described for treating cancer in patients in need thereof. In exemplary cases the patient has a cancer that has been linked to up-regulation of the HMTases G9a, SUV39H1, SUV39H2, MLL1, NSD2, or GLP. In some instances the cancer is leukemia. In other instances the cancer to be treated is colon cancer.

In some embodiments, methods and compositions are provided for treating cancer. Cancers that can be treated using the composition and methods describe herein include sarcomas, lymphomas, leukemias, carcinomas, blastomas, and germ cell tumors. A representative but non-limiting list of cancers that the disclosed compositions can be used to treat include lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, kidney cancer, lung cancers such as small cell lung cancer and non-small cell lung cancer, neuroblastoma/glioblastoma, ovarian cancer, pancreatic cancer, prostate cancer, skin cancer, liver cancer, melanoma, squamous cell carcinomas of the mouth, throat, larynx, and lung, colon cancer, cervical cancer, cervical carcinoma, breast cancer, epithelial cancer, renal cancer, genitourinary cancer, pulmonary cancer, esophageal carcinoma, head and neck carcinoma, large bowel cancer, hematopoietic cancers; testicular cancer; colon and rectal cancers, prostatic cancer, and pancreatic cancer. In preferred embodiments the cancer to be treated is associated with overexpression of one or more HMTases. In particularly preferred embodiments the one or more HMTases is selected from the group consisting of MLL1, NSD2, GLP, G9a, SUV39H1, and SUV39H2.

In preferred embodiments, a therapeutic amount of composition containing Verticillin A is co-administered with a therapeutic amount of a composition containing death receptor agonist, wherein Verticillin A reduces resistance of the cancer cells to the death receptor agonist. Verticillin A can inhibit G9a, GLP, SUV39H1, and SUV39H2 thereby reducing or inhibiting H3K9 methylation. Reduction in H3K9 methylation results in transcriptionally active chromatin to activate tumor suppressor genes.

C. Methods for Treating HIV

Human immunodeficiency virus (HIV) is a retrovirus that causes acquired immunodeficiency syndrome (AIDS). HIV type 1 (HIV-1) is the most virulent, most infective, and is the cause of the majority of HIV infections globally. A major obstacle to eradication of the virus in patients treated with potent antiretroviral therapies remains the persistence of latent HIV-infected cellular reservoirs. The virus latency refers to a period of inactivity during which virus production ceases but the virus genome has not been eradicated. In order to purge these viral reservoirs of latent HIV-1 virus, combination therapies that include a powerful HIV-1 antiretroviral as well as an active agent capable of antagonizing the latent virus in these reservoirs. G9a has been implicated as an important regulator for the maintenance of HIV-1 latency (Imai et al., Journal of Biological Chemistry, 285:16538-16545, 2010). The SUV39H1 MTase inhibitor chaetocin have been observed to induce 25-fold induction of latent HIV-1 virus without causing T cell activation (Bernhard, W. et al., FEBS Letters, 16,585:3549-3554, 2011). Therefore, in some instances compositions, formulations, and methods are provided for treating HIV-1 virus with Verticillin A. The Verticillin A can be administered in combination with one or more antiretrovirals to diminish or eradicate latent HIV-1 virus in a patent in need thereof.

In some embodiments methods and compositions are provided for treating HIV, in particular for treating and/or eradicating latent pools or reservoirs of HIV. In some cases methods and compositions are provided for induction of latent pools or reservoirs of HIV. The methods include providing to a patient carrying HIV a pharmaceutical formulation containing a therapeutically effective amount of Verticillin A. In some cases the therapeutically effective amount of Verticillin A is sufficient to produce a 10-fold induction in latent HIV-1 expression, preferably a 20-fold induction in latent HIV-1 expression, more preferably a 30-fold induction in latent HIV-1 expression. In preferred embodiments the Verticillin A provides induction of latent HIV virus while producing little to no T cell response. In other embodiments the formulation for treating HIV includes Verticillin A in combination with one or more HIV therapeutics or is provided in combination with one or more formulations containing one or more HIV therapeutics. As a non-limiting example, a pharmaceutical formulation containing Verticillin A, Zidovudine, and Lamivudine may be administered to a patient carrying HIV, particularly to a patient possessing latent pools of the virus. In an alternative example, a formulation containing Verticillin A is administered to a patient in combination with COMBIVIR®, which is a fixed dose formulation containing Zidovudine, and Lamivudine and marketed by GlaxoSmithKline. The two formulations may be administered at the same time or at different times.

In certain embodiments, a therapeutically effective amount of Verticillin A will be effective to result in a 2-fold induction of latent HIV virus, preferably a 5-fold induction of latent HIV-virus, more preferably a 10-fold induction of latent HIV virus.

D. Methods for Treating Asthma

Allergic asthma is the most common form of asthma, impacting more than half of the more than 20 million asthma sufferers. Asthma impacts more than 1 out of every 5 Americans. The symptomatic wheezing and labored breathing in allergic (atopic) asthma attacks is caused by an underlying inflammatory response of the T-helper 2 (Th2) cells. SUV39H1 inhibitors have been observed to destabilize or down-regulate the production of Th2 cells. The down-regulation of Th2 cells in allergic asthma patients will lead to decreased symptoms and decreased inflammatory response. It is therefore provided in certain circumstances compositions, formulations, and methods for using Verticillin A in the treatment of patients with allergic asthma.

In other instances methods and compositions are provided for treating allergic asthma. The method includes administering to a patient with allergic asthma a pharmaceutical formulation containing a therapeutically effective amount of Verticillin A. The formulation can optionally contain one or more conventional asthma therapeutics that may include corticosteroids, Leukotriene blockers, or long-acting beta agonist and may contain one or more additional active agents selected from the group consisting of bronchodilators, vasodilators, and mucokinetics.

In some embodiments the administration of Verticillin A results in a reduction in Th2 cells. In preferred embodiments the administration of Verticillin A results in a reduction or disappearance of one or more symptoms associated with allergic asthma, including reducing or eliminating wheezing, reducing or eliminating inflammation of the airways, and provide for improved and less labored breathing.

E. Methods for Treating Progeria and Age-Related Disorders

Progeria is a rare genetic condition closely related to normal human aging. Progeria occurs in young children and results in rapid aging leading to a range of complications and to early death (typically within the first twenty years of life). There is currently no specific treatment for progeria. Progeria is closely tied to normal cellular senescence, whereby normal diploid cells lose the ability to divide normally. At least in vitro, cellular senescence has been observed to be correlated with H3K9 methylation (catalyzed by SUV39H1 HMTase) (Narita M, Nunez S, Heard E, Narita M, Lin A W, Hearn S A, Spector D L, Hannon G J, Lowe S W. 2003. Rb-mediated heterochromatin formation and silencing of E2F target genes during cellular senescence. Cell 113: 703-716). In a mouse model of progeria, depletion of SUV39H1 HMTase rescues defective DNA repair and extends lifespan. SUV39H1 inhibitors are therefore interesting targets for the treatment of progeria and disorders related to cellular senescence. Compositions, formulations, and methods are therefore provided for treating progeria and slowing the progression of cellular senescence with Verticillin A.

In additional embodiments methods and compositions are provided for treating age-related disorders, and in particular for treating and diminishing the progression of progeria. The method includes administering to a patient with an age-related disorder or progeria a pharmaceutical formulation containing a therapeutically effective amount of Verticillin A. In preferred embodiments the administration of Verticillin A results in increased levels of comfort and/or increased life expectancy for subjects with progeria.

### VI. Definitions

The term “co-administration” includes simultaneous and sequential administration. An appropriate time course for sequential administration may be chosen by the physician, according to such factors as the nature of a patient's illness, and the patient's condition.

The term “inhibit,” “inhibiting,” or “inhibition” refers to a decrease in activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

The term “subject” refers to any individual who is the target of administration. The subject can be a vertebrate, for example, a mammal. Thus, the subject can be a human. The term does not denote a particular age or sex. The term “patient” refers to a subject afflicted with a disease or disorder. The term “patient” includes human and veterinary subjects.

The term “target cell” refers to a cell bearing the targeted death receptor, including, for example, a cell that expresses DR5 or DR4. Preferably, the target cell is an abnormally growing cell or tumor cell.

The term “therapeutically effective” means that the amount of the composition used is of sufficient quantity to ameliorate one or more causes or symptoms of a disease or disorder. Such amelioration only requires a reduction or alteration, not necessarily elimination. For example, a therapeutically effective amount of a composition containing a death receptor agonist is the quantity sufficient to cause apoptosis in one or more target cells. As used herein, the terms “therapeutically effective amount” “therapeutic amount” and “pharmaceutically effective amount” are synonymous. One of skill in the art could readily determine the proper therapeutic amount.

The term “treat” or “treatment” refers to the medical management of a subject with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

The term “prevent,” “preventing,” or “prevention” does not require absolute forestalling of the condition or disease but can also include a reduction in the onset or severity of the disease or condition. For example, in the case of death receptor resistance, to prevent a target cell's resistance to a death receptor agonist is to make the cell less resistant to said agonist.

The term “pharmaceutically acceptable salt”, as used herein, refer to derivatives of the compounds defined herein, wherein the parent compound is modified by making acid or base salts thereof. Example of pharmaceutically acceptable salts include but are not limited to mineral or organic acid salts of basic residues such as amines; and alkali or organic salts of acidic residues such as carboxylic acids. The pharmaceutically acceptable salts include the conventional non-toxic salts or the quaternary ammonium salts of the parent compound formed, for example, from non-toxic inorganic or organic acids. Such conventional non-toxic salts include those derived from inorganic acids such as hydrochloric, hydrobromic, sulfuric, sulfamic, phosphoric, and nitric acids; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, tolunesulfonic, naphthalenesulfonic, methanesulfonic, ethane disulfonic, oxalic, and isethionic salts.

The pharmaceutically acceptable salts of the compounds can be synthesized from the parent compound, which contains a basic or acidic moiety, by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, ethanol, isopropanol, or acetonitrile are preferred. Lists of suitable salts are found in Remington's Pharmaceutical Sciences, 20th ed., Lippincott Williams & Wilkins, Baltimore, Md., 2000, p. 704; and “Handbook of Pharmaceutical Salts: Properties, Selection, and Use,” P. Heinrich Stahl and Camille G. Wermuth, Eds., Wiley-VCH, Weinheim, 2002.

As generally used herein “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

Unless defined otherwise, all technical and scientific terms used herein have the same meanings as commonly understood by one of skill in the art to which the disclosed invention belongs. Publications cited herein and the materials for which they are cited are specifically incorporated by reference.

### EXAMPLE 1

### Inhibitory Activity of Verticillin A for Known Methyltransferases

Verticillin A was tested as an inhibitor against 18 known methyltransferases. (S-(5′-Adenosyl)-L-homocysteine) (SAH) was used as a control. Verticillin A was tested in 10-dose IC₅₀ mode with 3-fold serial dilution starting at 10 μM concentration. SAH was tested in 10-dose IC₅₀ modes with 3-fold serial dilution starting at 100 μM concentration. All reactions were carried out with a S-adenosyl methionine (SAM) concentration of 1 μM.

The methyltransferases tested included the human DNA (cytosine-5) methyltransferases DNMT3a and BNMT3b; the non-SET domain containing H3K79 methyltransferase DOT 1; the H3K27 methyltransferase EZH2; the Mixed-Lineage Leukemia H3K4Me methyltransferases MLL1, MLL2, MLL3, and MLL4; the probable histone-lysine N-methyltransferase NSD2 (also known as Wolf-Hirschhorn syndrome candidate 1 protein, WHSC1); the protein arginine N-methyltransferases PRMT1, PRMT3, PRMT4, PRMT5, and PRMT6; the H3K4 specific methyltransferase SET7; the H4L20 specific methyltransferase SET8 (also known as PR-SETT); the SET domain and mariner transposase fusion gene-containing protein SETMAR; the N-lysine methyltransferase SMYD2; the histone-lysine N-methyltransferase EZH1; the methyltransferase G9a that can methylate K9 and K27 of H1 and H3; the G9a-like methyltransferase GLP; and the H3K9 selective methyltransferases SUV39H1 and SUV39H2.

TABLE 1
Measured IC₅₀ (M) values for Verticillin A and SAH at 1
μM SAM concentration. Empty cells indicate no inhibition or
an activity that could not be fit to an IC₅₀ curve.

| Methyltransferase | Verticillin A IC₅₀ (M) | SAH IC₅₀ (M) |
|---|---|---|
| DNMT3a | | 1.99E−07 |
| DNMT3b | | 2.23E−07 |
| DOT1 | | 1.99E−06 |
| EZH2 | | 4.68E−05 |
| MLL1 | 3.08E−06 | 2.36E−06 |
| MLL2 | | 1.31E−05 |
| MLL3 | | 9.69E−06 |
| MLL4 | | 7.61E−06 |
| NSD2 | 4.13E−06 | 5.05E−06 |
| PRMT1 | | 3.40E−07 |
| PRMT3 | | 1.59E−06 |
| PRMT4 | | 3.58E−07 |
| PRMT5 | | 7.75E−07 |
| PRMT6 | | 2.95E−07 |
| SET7 | | 3.35E−05 |
| SET8 | | >1.00E−04 |
| SETMAR | | 3.07E−06 |
| SMYD2 | | 1.22E−06 |
| EZH1 | | >1.00E−04 |
| G9a | 5.38E−07 | 4.45E−06 |
| GLP | 1.27E−06 | 3.45E−07 |
| SUV39H1 | 5.73E−07 | >1.00E−04 |
| SUV39H2 | 4.81E−07 | 5.73E−05 |

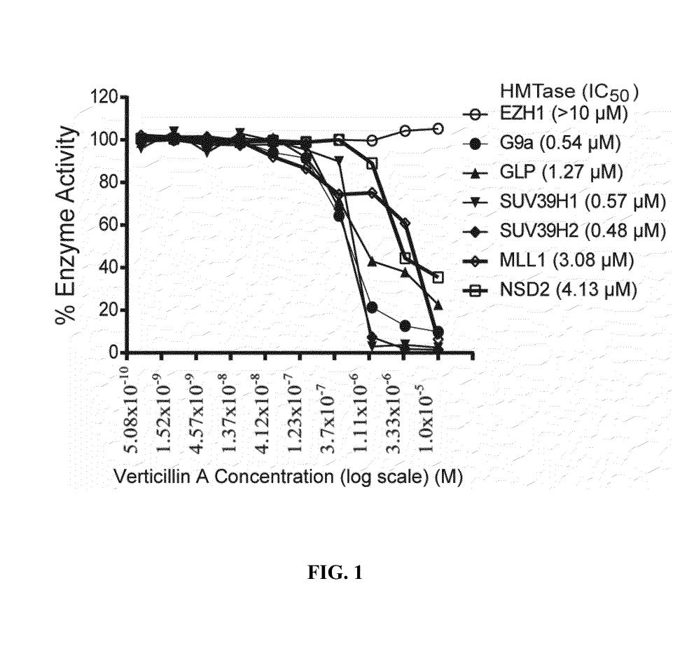

Verticillin A was observed to have potent inhibitory activity for the enzymes G9a (IC₅₀ of 0.54 μM), SUV39H1 (IC₅₀ of 0.57 μM), and SUV39H2 (IC₅₀ of 0.48 μM). Verticillin A was observed to also exhibit inhibitory activity against MLL1 (IC₅₀ of 3.08 μM), NSD2 (IC₅₀ of 4.13 μM), and GLP (IC₅₀ of 1.27 μM). FIG. 1 depicts a plot of the enzyme activity (as a percentage of enzyme activity relative to activity in DMSO) versus the log of the Verticillin A concentration for G9a, GLP, SUV39H1, SUV39H2, MLL1, NSD2, as well as for EZH1 for which no inhibitory activity was observed.

In some embodiments methods are provided for modifying gene expression using Verticillin A by inhibiting or reducing methylation of DNA-binding proteins. In some instances one or more histone methyltransferases are contacted with an effective amount of Verticillin A to inhibit the one or more histone methyltransferases. Preferred histone methyltransferases are selected from the group consisting of G9a, GLP, SUV39H1, SUV39H2, MLL1, and NSD2.

### EXAMPLE 2

### In-Vivo Inhibitory Activity of Verticillin A in Human Colon Carcinoma Cells

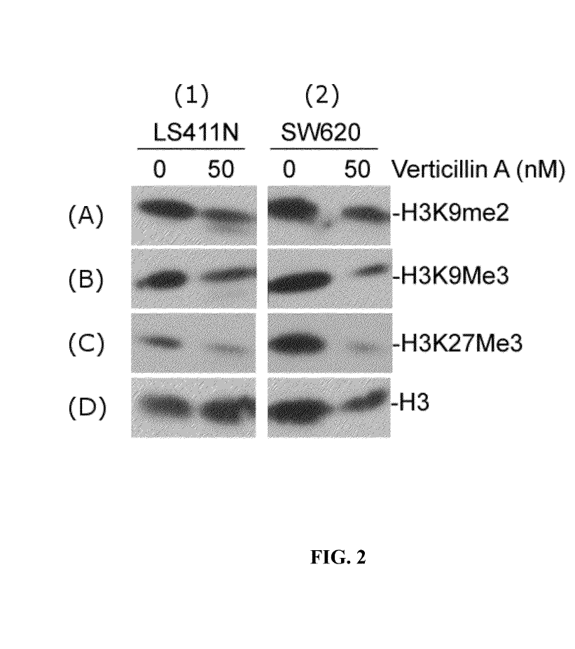

Verticillin A was tested as a HMTase inhibitor in human colon carcinoma cells (LS411N and SW620) in vivo. Western blotting analysis was used to test the levels of H3K9Me2, H3K9Me3, and H3K27Me3 in LS411N and SW620 cells after exposure to 50 nM concentration of Verticillin A. The results are presented in FIG. 2. The results demonstrate that levels of H3K9Me2, H3K9Me3, and H3K27Me3 are reduced in LS411N and SW620 cells upon exposure to 50 nM Verticillin A.A 50 nM dose of Verticillin was observed to effectively inhibit H3K9Me2, H3K9Me3 and H3K27Me3, indicating that Verticillin A is a potent and selective HMTase inhibitor in vivo.

Those skilled in the art will recognize, or be able to ascertain using no more than routine experimentation, many equivalents to the specific embodiments of the invention described herein. Such equivalents are intended to be encompassed by the following claims.

### EXAMPLE 3

### Verticillin A-Induced Global Gene Expression Profiles

5-FU-resistant human metastatic colon carcinoma LS411N-5FU-R cells were treated with verticillin A (50 nM) for 3 days. RNA was isolated from untreated and treated cells and analyzed for gene expression profiles using Affymetrix gene chip. The genes whose expression levels were altered (either down-regulated or uo-regulated) more than 1.6 folds by verticillin A are shown below.

Biological Process #genes (% total number of genes)

apoptosis (GO:0006915) 46 (5.9%).

cell adhesion (GO:0007155) 55 (7.1%)

cell communication (GO:0007154) 192 (24.7%)

cell cycle (GO:0007049) 90 (11.6%)

cellular component organization (GO:0016043) 50 (6.4%)

cellular process (GO:0009987) 283 (36.4%)

developmental process (GO:0032502) 132 (17%)

generation of precursor metabolites and energy (GO:0006091) 18 (2.3%)

homeostatic process (GO:0042592) 5 (0.6%)

immune system process (GO:0002376) 133 (17.1%)

localization (GO:0051179) 4 (0.5%)

metabolic process (GO:0008152) 400 (51.5%)

regulation of biological process (GO:0050789) 4 (0.5%)

reproduction (GO:0000003) 42 (5.4%)

response to stimulus (GO:0050896) 93 (12%)

system process (GO:0003008) 88 (11.3%)

transport (GO:0006810) 86 (11.1%)

We claim:

1. A method of inhibiting a histone methyltransferase comprising contacting the histone methyltransferase with an effective amount of Verticillin A.

2. A method of inhibiting a histone methyltransferase in a subject in need thereof comprising administering to the subject a pharmaceutical formulation containing a therapeutically effective amount of Verticillin A.

3. The method of claim 1 wherein the histone methyltransferase is selected from the group consisting of MLL1, NSD2, GLP, G9a, SUV39H1, and SUV39H2.

4. The method of claim 2 wherein the formulation contains one or more additional active agents.

5. The method of claim 4 wherein the one or more additional active agents is selected from the group consisting of histone acetyltransferases, histone deacetylases, histone acetyltransferase inhibitors, histone deacetylase inhibitors, histone methyltransferases, histone demethylases, histone methyltransferase inhibitors, and histone demethylase inhibitors.

6. The method of claim 2 wherein the subject in need thereof has HIV.

7. The method of claim 6 wherein the formulation contains one or more HIV therapeutics.

8. The method of claim 6 further comprising administering a second pharmaceutical formulation containing one or more HIV therapeutics.

9. The method of claim 7 wherein the one or more HIV therapeutics is selected from the group consisting of HIV protease inhibitors, HIV nucleoside and HIV nucleotide reverse transcriptase inhibitors, HIV non-nucleoside reverse transcriptase inhibitors, entry inhibitors, and maturation inhibitors.

10. The method of claim 7 wherein the formulation containing Verticillin A is administered as part of HAART treatment.

11. The method of claim 2 wherein the individual in need thereof has asthma.

12. The method of claim 11 wherein the formulation contains one or more asthma therapeutics.

13. The method of claim 11 further comprising administering a second pharmaceutical formulation containing one or more asthma therapeutics.

14. The method of claim 12 wherein the one or more asthma therapeutics is selected from the group consisting of corticosteroids, leukotriene modifiers, and long-acting beta agonists.

15. The method of claim 11 wherein the formulation is in the form of a particle, liquid, or aerosol for pulmonary administration.

16. The method of claim 2 wherein the individual in need thereof has an age-related disease or disorder.

17. The method of claim 16 wherein the disease or disorder is progeria.

18. A pharmaceutical formulation comprising a therapeutically effective amount of Verticillin A and one or more additional active agents selected from the group consisting of HIV protease inhibitors, HIV nucleoside inhibitors, HIV nucleotide reverse-transcriptase inhibitors, HIV non-nucleoside reverse transcriptase inhibitors, corticosteroids, leukotriene modifiers or leukotriene blockers, long-acting beta agonist, bronchodilators, vasodilators, and mucokinetics.
